(19) United States
(12) Patent Application Publication
(10) Pub. No.: US 2026/0055098 A1
(43) Pub. Date: Feb. 26, 2026

(54) TOLFENPYRAD DERIVATIVES EXHIBIT POTENT FRANCISELLA-SPECIFIC ANTIBACTERIAL ACTIVITY WITHOUT TOXICITY TO MAMMALIAN CELLS IN VITRO

(71) Applicant: University of Florida Research Foundation, Incorporated, Gainesville, FL (US)

(72) Inventor: Aria Eshraghi, Gainesville, FL (US)

(21) Appl. No.: 19/305,073

(22) Filed: Aug. 20, 2025

Related U.S. Application Data
(60) Provisional application No. 63/685,368, filed on Aug. 21, 2024.

(51) Int. Cl. C07D 417/14; A61K 31/40; A61K 31/4245; A61K 31/427; A61K 31/433; A61K 31/4439; A61P 31/04; C07D 207/34; C07D 413/12; C07D 417/12

(52) U.S. Cl. CPC C07D 417/14; A61K 31/40; A61K 31/4245; A61K 31/427; A61K 31/433; A61K 31/4439; A61P 31/04; C07D 207/34; C07D 413/12; C07D 417/12

(57) ABSTRACT

The subject matter described herein is directed to tolfenpyrad derivatives of Formula I, or pharmaceutically acceptable salts thereof, which exhibit antibacterial activity against Francisella species with little to no toxicity to mammalian cells. Pharmaceutical compositions of tolfenpyrad derivatives, and methods of treating disorders associated with Gram-negative intracellular bacterial pathogens, such as Francisella tularensis, are also described.

### CROSS-REFERENCE TO RELATED APPLICATIONS

This application claims the benefit of priority to U.S. Provisional Patent Application No. 63/685,368, filed on Aug. 21, 2024, the content of which is incorporated by reference herein in its entirety for all purposes.

### BACKGROUND

Bacterial species of the genus Francisella are a deadly group of pathogens that cause the emerging disease tularemia. Discovering novel antibiotics that target Francisella species is central to safeguarding public health. Francisella tularensis is a Gram-negative intracellular bacterial pathogen with the potential to infect humans and animals with as few as 10 viable bacteria. Owing to its ease of dissemination through aerosols, expected high mortality rates, and potential for major public health impact and social disruption, the Centers for Disease Control and Prevention has categorized F. tularensis as an agent of greatest concern for use as a potential weapon of bioterrorism. This pathogen can infect a variety of phagocytic cells by employing a poorly-characterized type VI secretion system to traffic through the endosomal network and into the cytosol, where it replicates and causes the highly lethal disease tularemia. In humans, tularemia is most commonly associated with the F. tularensis subspecies tularensis (type A) and holarctica (type B). Type A strains cause life-threatening septicemia and are mainly restricted to North America. In contrast, type B strains are found throughout Europe and Asia, and often cause a milder form of tularemia. The form of this disease and its severity depend on the route of entry and the subspecies of pathogen, with clinical signs presenting as ulceroglandular, oculoglandular, oropharyngeal, typhoidal, and/or pneumonic forms. In recent years, there has been a substantial increase in the emergence of tularemia infections in Europe. For example, a four- to ten-fold increase in the incidence of tularemia has been recorded in Switzerland and Sweden, highlighting the cause for growing public health concern.

Although Francisella species are intrinsically resistant to beta-lactams and some strains exhibit resistance to macrolides. Currently, most infections are treated with aminoglycosides, fluoroquinolones, and tetracyclines. However, the potential development of antibiotic-resistant strains poses a threat to public health security. Several studies have demonstrated that resistance to various antibiotics can be rapidly developed through facile methods. For this reason, the is a need for new and improved antibiotics.

The present application relates to tolfenpyrad derivatives which act as novel antibiotic compounds with improved efficacy against F. novicida while harboring little to no toxicity to mammalian cells.

### BRIEF SUMMARY

Described are tolfenpyrad derivatives of Formula I, or pharmaceutically acceptable salts or stereoisomers thereof:

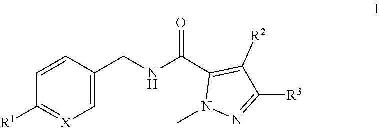

wherein:
X is CH or N;
R¹ is independently selected from the group consisting of C₁-C₁₀ alkyl, C₁-C₁₀ haloalkyl, C₃-C₁₀ cycloalkyl, C₁-C₁₀ alkoxy, 3-10 membered heterocycloalkyl, aryl, aryloxy, and heteroaryl;
R² and R³ are each independently selected from hydrogen, halogen, C₁-C₁₀ alkyl, C₂-C₁₀ alkenyl, C₂-C₁₀ alkynyl, C₁-C₁₀ haloalkyl, C₃-C₁₀ cycloalkyl, 4-10 membered heterocycloalkyl, aryl, aryloxy, and heteroaryl;
or,
R² and R³ are taken together with the carbons to which they are attached to form a C₃-C₁₀ cycloalkyl, 4-10 membered heterocycloalkyl, or heteroaryl; and,
R¹ cannot be

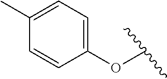 [SMILES: *Oc1ccc(C)cc1]

when R² is Cl and R³ is C₁-C₁₀ alkyl.

Also described are pharmaceutical compositions comprising any of the tolfenpyrad derivatives described herein, and methods of treatment of a disorder associated with a Francisella species. In some embodiments, the Francisella species is F. novicida. In some embodiments, the methods comprise administering a subject suffering from the disorder an effective amount of at least one tolfenpyrad derivative or a pharmaceutical composition comprising at least one tolfenpyrad derivative as described herein.

### BRIEF DESCRIPTION OF THE FIGURES

FIG. 1A-D shows graphs illustrating identification of tolfenpyrad derivatives that block F. novicida growth. (A) The structure of tolfenpyrad with pyrazole-5-carboxamide and p-methylphenoxybenzyloxy moieties indicated, along with the sites of modification of the left side (LS), middle region (MR) and right side (RS). (B) Dose-response curve of F. novicida grown in a titration of tolfenpyrad, with vertical dashed lines indicating 0.75 μM and 2 μM. (C and D) Growth of F. novicida in the presence of 0.75 μM (C) and 2 μM (D) tolfenpyrad and its derivatives as compared to untreated bacteria in late exponential phase of growth, which are set to 100%. The horizontal dashed lines represent arbitrary thresholds set at 60% and 80% inhibition.

FIG. 1E shows comparison of dose-response curves of F. novicida growth when treated with a titration of tolfenpyrad and the described tolfenpyrad derivatives. From left to right, slopes at 40% F. novicida growth correspond to: NK06, NK05, NK01, NK02, NK07, NK11, NK03, NK08, NK10, NK09, NK04, NK12, and tolfenpyrad.

FIG. 2A-C shows graphs illustrating the evaluation of drug-induced macrophage toxicity to identify tolfenpyrad derivatives that are less toxic and more effective than tolfenpyrad. (A) The viability of immortalized bone marrow derived macrophages (iBMDM) incubated for 16 h with a titration of tolfenpyrad in DMEM or DMEM lacking glucose (DMEM-Glucose), as measured by leakage of lactate dehydrogenase into the culture supernatant. (B) Viability of iBMDM cells incubated with 2 μM drugs for 16 hours in DMEM-Glucose (tolfenpyrad arrow). (C) Viability of iBMDM cells graphed against the growth of F. novicida in the presence of 2 μM tolfenpyrad and various tolfenpyrad derivatives (data in FIG. 3B and FIG. 1D, respectively). Tolfenpyrad showed 7.8% Macrophage viability and 31.4% F. novicida growth, NK03 showed 79.5% Macrophage viability and 17.8% F. novicida growth, NK07 showed 94.0% Macrophage viability and 8.1% F. novicida growth, and NK11 showed 113.0% Macrophage viability and 10.9% F. novicida growth.

FIG. 2D-G shows effects of tolfenpyrad and tolfenpyrad derivatives NK03, NK07, and NK11 on F. novicida growth inhibition and on iBMDM cell toxicity. Data from separate experiments to enumerate the viability of iBMDM cells in DMEM-Glucose (lines with square points) and growth of F. novicida (lines with circle points) incubated with titrations of tolfenpyrad (D), NK03 (E), NK07 (F), and NK11 (G).

FIG. 3A-C shows tolfenpyrad derivatives inhibit F. novicida pathogenicity in vitro and in vivo. (A) Growth of F. novicida in iBMDM cells cultured in DMEM and treated with tolfenpyrad or derivatives NK03, NK07, and NK11. Intracellular bacteria growth was analyzed after 16 h and normalized to the number of bacteria at T=0. The horizontal dotted line represents intramacrophage growth of F. novicida in untreated iBMDM cells. From left to right, slopes at 10² Intramachrophage growth correspond to NK07, NK11, NK03, and tolfenpyrad. (B) The viability of iBMDM cells in DMEM treated with titrations of tolfenpyrad, NK03, NK07, and NK11 for 16 h. The horizontal dotted line represents the viability of untreated iBMDM cells. Lines, at 0.63 μM, from top to bottom, correspond to: NK07, NK03, NK11, and tolfenpyrad. (C) Kaplan-Meier curve indicating survival of G. mellonela larvae infected with 10⁵ CFU of F. novicida and subsequently treated with 80 μM tolfenpyrad or NK11. Control groups received PBS or F. novicida followed by PBS injection instead of tolfenpyrad or tolfenpyrad derivatives. Statistical comparison was performed with Log-rank (Mantel-Cox) test.

FIG. 3D shows survival of G. mellonella larvae injected with PBS, followed by injection of PBS or 100 μM of tolfenpyrad or NK11.

FIG. 4 shows tolfenpyrad-selected strains of F. novicida are resistant to derivatives. Dose response curves of tolfenpyrad (A), NK03 (B), NK07 (C), and NK11 (D) on wild-type F. novicida (line A), 3×PM (OsrR G54D, P68S, A216V) (line B), and A_10 (OsrR G54D, NuoM M252I) (line C).

FIG. 5 shows viability of an F. novicida culture before and after treatment with 10 μM of tolfenpyrad, NK07, NK03, and NK11, indicating inhibition of F. novicida growth through a bacteriostatic mechanism. Tolfenpyrad and derivatives were added two hours after the start of the experiment (arrow) and viable bacteria were counted via determination of colony forming units. Lines, from top to bottom line at 5 h, correspond to: untreated, NK11, NK03, tolfenpyrad, and NK07.

FIG. 6 shows P. aeruginosa is resistant to tolfenpyrad and tolfenpyrad derivatives. The growth of P. aeruginosa in the presence of 30 μM of tolfenpyrad (arrow) and tolfenpyrad derivatives, as measured by recording OD_{600 nm} when untreated bacteria reached late log phase. Untreated controls are set to 100%.

FIG. 7 shows drug-induced macrophage toxicity is minimal in DMEM. The viability of iBMDM cells incubated with 2 μM tolfenpyrad (arrow) and tolfenpyrad derivatives for 16 h in DMEM. Untreated controls are set to 100%.

### DETAILED DESCRIPTION

Species within the Francisella genus are remarkably virulent, with the most pathogenic strains causing ≥60% mortality with exposure to as few as 10 viable bacteria. Francisella species are intrinsically resistant to several antibiotics, including beta-lactams, polymyxins, glycopeptides, sulfonamides and trimethoprim. Tolfenpyrad is an insecticide used worldwide that has been observed to inhibit the growth of species within the Francisella genus. Tolfenpyrad also exhibits a broad biological activity against many eukaryotes, including fungi, ticks, mites, and nematodes. Interestingly, the antibacterial activity of tolfenpyrad is restricted to Francisella species, as other Gram-negative and Gram-positive bacteria are resistant.

The present disclosure describes tolfenpyrad derivatives having improved antibacterial activity and reduced mammalian toxicity. The structure-activity relationship of 262 tolfenpyrad derivatives were investigated. By measuring the activity of these compounds and their safety toward host cells in vitro, derivatives were identified that significantly improved anti-Francisella activity while reducing toxicity to macrophages. Described are tolfenpyrad derivatives that are active against human-virulent strains of Francisella, block intramacrophage growth of F. novicida, and have reduced F. novicida-induced mortality relative to tolfenpyrad in an invertebrate in vivo model for tularemia.

Described are tolfenpyrad derivatives of Formula I,

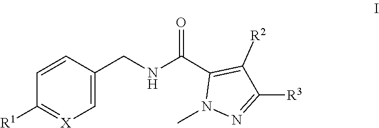

wherein:
X is CH or N;
R¹ is independently selected from the group consisting of C₁-C₁₀ alkyl, C₁-C₁₀ haloalkyl, C₃-C₁₀ cycloalkyl, C₁-C₁₀ alkoxy, 3-10 membered heterocycloalkyl, aryl, aryloxy, and heteroaryl;
R² and R³ are each independently selected from hydrogen, halogen, C₁-C₁₀ alkyl, C₂-C₁₀ alkenyl, C₂-C₁₀ alkynyl, C₁-C₁₀ haloalkyl, C₃-C₁₀ cycloalkyl, 4-10 membered heterocycloalkyl, aryl, aryloxy, and heteroaryl;
or,
R² and R³ are taken together with the carbons to which they are attached to form a C₃-C₁₀ cycloalkyl, 4-10 membered heterocycloalkyl, or heteroaryl; and,
R¹ cannot be

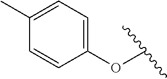 [SMILES: *Oc1ccc(C)cc1]

when R² is Cl and R³ is C₁-C₁₀ alkyl.

The tolfenpyrad derivatives also include pharmaceutically acceptable salts or stereoisomers of the compounds of formula I.

The presently described subject matter will now be described more fully.

### I. Definitions

Unless otherwise defined, all terms (including technical and scientific terms) used herein have the same meaning as commonly understood by one of ordinary skill in the art to which this subject matter belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the subject matter herein is for the purpose of describing particular aspects only and is not intended to be limiting of the subject matter. In case of a conflict in terminology, the present specification is controlling.

The term “alkyl” refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. The term “alkyl” is intended to include both substituted and unsubstituted alkyl unless otherwise indicated. Substitutions include, but are not limited to, halo, alkyl, haloalkyl, cycloalkyl, aryl, heterocycloalkyl, hydroxyl, alkoxy, haloalkoxy, cycloalkoxy, aryloxy, mercapto, amino, carboxy, ester, amide, sulfonamide, nitro or cyano.

The terms “amino” or “amine” are represented by the formula NA¹A², where A¹ and A² are each independently hydrogen, alkyl, haloalkyl, cycloalkyl, alkenyl, alkynyl, aryl, and heteroaryl.

The term “cycloalkyl” refers to a saturated or partially unsaturated cyclic hydrocarbon group containing from 3-10 carbons which may be a single ring or multiple rings wherein the multiple rings may be fused, bridged or spiro, and may comprise one or more (e.g., 1 to 3) oxo (═O) moieties. Representative examples of cycloalkyl include, cyclopropyl, cyclobutyl, cyclopentyl, cyclohexyl, cycloheptyl, and cyclooctyl. The “cycloalkyl” group may be optionally substituted with one or more alkenyl, alkynyl, halo, alkyl, haloalkyl, cycloalkyl, aryl, heterocycloalkyl, hydroxyl, alkoxy, haloalkoxy, cycloalkoxy, aryloxy, mercapto, amino, carboxy, ester, amide, sulfonamide, nitro or cyano.

The term “heterocycloalkyl” refers to a saturated or partially unsaturated cyclic hydrocarbon group containing from 3-10 carbons and which has at least one heteroatom (O, N, or S) incorporated within the ring. The “heterocycloalkyl” may be a single ring or multiple rings wherein the multiple rings may be fused, bridged or spiro, and may comprise one or more (e.g., 1 to 3) oxo (═O) moieties. The “heterocycloalkyl” group may be optionally substituted with one or more alkenyl, alkynyl, halo, alkyl, haloalkyl, cycloalkyl, aryl, heterocycloalkyl, hydroxyl, alkoxy, haloalkoxy, cycloalkoxy, aryloxy, mercapto, amino, carboxy, ester, amide, sulfonamide, nitro or cyano.

The term “aryl” refers to a monocyclic carbocyclic ring system or a bicyclic carbocyclic fused ring system having one or more aromatic rings. Representative examples of aryl include, azulenyl, indanyl, indenyl, naphthyl, phenyl, tetrahydronaphthyl, and the like. The “aryl” group may be optionally substituted with one or more alkenyl, alkynyl, halo, alkyl, haloalkyl, cycloalkyl, aryl, heterocycloalkyl, hydroxyl, alkoxy, haloalkoxy, cycloalkoxy, aryloxy, mercapto, amino, carboxy, ester, amide, sulfonamide, nitro or cyano.

The term “heteroaryl” refers to monocyclic- or bicyclic-ring system that contains any aromatic group that has at least one heteroatom incorporated within the ring of the aromatic group. The “heteroaryl” group may be optionally substituted with one or more alkenyl, alkynyl, halo, alkyl, haloalkyl, cycloalkyl, aryl, heterocycloalkyl, hydroxyl, alkoxy, haloalkoxy, cycloalkoxy, aryloxy, mercapto, amino, carboxy, ester, amide, sulfonamide, nitro or cyano.

The term “alkoxy” refers to an alkyl group, as defined herein, appended to the parent molecular moiety through an oxy group, —O—. Representative examples of alkoxy include, but are not limited to, methoxy, ethoxy, propoxy, 2-propoxy, butoxy, tert-butoxy, pentyloxy, hexyloxy and the like.

The term “alkenyl” refers to a hydrocarbon having from 2 to 10 carbons with a structural formula containing at least one carbon-carbon double bond. Asymmetric structures are intended to include both the E and Z isomers. The “alkenyl” group may be optionally substituted with one or more halo, alkyl, haloalkyl, cycloalkyl, aryl, heterocycloalkyl, hydroxyl, alkoxy, haloalkoxy, cycloalkoxy, aryloxy, mercapto, amino, carboxy, ester, amide, sulfonamide, nitro or cyano.

The term “alkynyl” means a hydrocarbon group of 2 to 10 carbon atoms with a structural formula containing at least one carbon-carbon triple bond. The “alkynyl” group may be optionally substituted with one or more halo, alkyl, haloalkyl, cycloalkyl, aryl, heterocycloalkyl, hydroxyl, alkoxy, haloalkoxy, cycloalkoxy, aryloxy, mercapto, amino, carboxy, ester, amide, sulfonamide, nitro or cyano.

The term “haloalkyl” refers to a straight or branched chain hydrocarbon (fully or partially saturated) containing from 1 to 10 carbon atoms where one or more hydrogen atoms are individually replaced by a halogen atom (F, Cl, Br, or I). The “haloalkyl” group may be optionally substituted with one or more cycloalkyl, aryl, heterocycloalkyl, hydroxyl, alkoxy, haloalkoxy, cycloalkoxy, aryloxy, mercapto, amino, carboxy, ester, amide, sulfonamide, nitro or cyano.

The term “aryloxy” refers to an aryl group, as defined herein, appended to the parent molecular moiety through an oxy group, —O—. Representative examples of aryloxy include, but are not limited to, phenoxy, naphthoxy, and the like.

The term “halo” or “halogen” refers to F, Cl, Br, or I.

The term “in vitro” refers to artificial environments and to processes or reactions that occur within an artificial environment (e.g., a test tube).

The term “in vivo” refers to natural environments (e.g., a cell or organism or body) and to processes or reactions that occur within a natural environment.

A “derivative” or “tolfenpyrad derivative” is a compound that has a three-dimensional structure that is similar to at least a part of the parent compound. In some embodiments, a derivative is a compound that is derived from, or imagined to derive from, a parent compound such as by substitution of at least one atom or group with another atom or group. In some embodiments, derivatives are compounds that at least theoretically can be formed from a common precursor compound.

“Optional” or “optionally” means that the subsequently described event or circumstance may but need not occur, and that the description includes instances where the event or circumstance occurs and instances in which it does not. For example, “aryl group optionally substituted with an alkyl group” means that the alkyl may but need not be present, and the description includes situations where the aryl group is substituted with an alkyl group and situations where the aryl group is not substituted with alkyl.

A “subject” or “patient” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees and apes, and, in particular, humans. In some embodiments, the subject can be human. In some embodiments, the subject can be a human child and/or a human infant, for example, a child or infant with a fever. In other embodiments, the subject can be a human adult.

The terms “treat” and “treatment” refer to both therapeutic treatment and prophylactic or preventative measures, wherein the object is to prevent or slow down (lessen) an undesired physiological change, disorder or adverse health condition. For purposes of this disclosure, beneficial or desired clinical results include, but are not limited to, alleviation of one or more symptoms, diminishment of extent of the condition, stabilized (i.e., not worsening) state of the condition, delay or slowing of progression of the condition, amelioration or palliation of the condition, and absence of condition (whether partial or total). Those in need of treatment include those already with the condition or disorder, diagnosed with the condition or disorder, as well as those prone to have the condition or disorder or those in which the condition or disorder is to be prevented.

A “therapeutically effective amount” or “effective amount” and the like refers to the amount of a compound of the present disclosure or a pharmaceutically acceptable salt thereof that, elicits the biological or medicinal response indicated. For example, when administered to a subject for treating a disease, the therapeutically effective amount of a compound is sufficient to prevent, alleviate or ameliorate one or more symptoms of disease or prolong the survival of the subject being treated. This response may occur in a tissue, system, animal or human and includes alleviation of the signs or symptoms of the disease being treated. Determination of an effective amount is well within the capability of those skilled in the art, in view of the disclosure provided herein. The “therapeutically effective amount” of the compounds described herein will vary depending on the compound, the disease and its severity and the age, weight, etc., of the subject to be treated. Designation of a range of values includes all integers within or defining the range, and all subranges defined by integers within the range.

Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, pharmaceutically acceptable includes compounds Generally Recognized as Safe (GRAS), approved by a regulatory agency of the Federal or state government, or listed in the U.S. Pharmacopeia or other generally recognized pharmacopeia for use in animals, and more particularly in humans.

The terms “salt(s)” and “pharmaceutically acceptable salts” denote salts which are not biologically or otherwise undesirable. Pharmaceutically acceptable salts include both acid and base addition salts. The phrase “pharmaceutically acceptable salt,” as used herein, refers to pharmaceutically acceptable organic or inorganic salts of a molecule. A pharmaceutically acceptable salt may involve the inclusion of another molecule that acts as a counterion. The counterion may be any organic or inorganic moiety that stabilizes the charge on the parent compound. Furthermore, a pharmaceutically acceptable salt may have more than one charged atom in its structure. Hence, a pharmaceutically acceptable salt can have one or more charged atoms and/or one or more counterions. Such salts may include quaternary ion complexes. Other suitable salts may include sulfate, tartrate, citrate, oxalate, phosphate, among others.

The terms “administer” and “administering” refer to a method of delivering agents, compounds, or compositions to the desired site of biological action. These methods include, but are not limited to, oral delivery, topical delivery, parenteral delivery, intravenous delivery, intradermal delivery, intramuscular delivery, intrathecal delivery, colonic delivery, rectal delivery, or intraperitoneal delivery.

The terms “about” and “approximately,” when used to modify an amount specified in a numeric value or range, indicates the numeric value as well as reasonable deviations from the value known to the skilled person in the art. In some embodiments, the term “about” means within the typical ranges of tolerances in the art. In some embodiments, the term “about” encompasses values within a standard margin of error of measurement (e.g., SEM) of a stated value or variations±0.5%, ±1%, ±5%, or ±10% from a specified value. When the term “about” is present before a series of numbers or a range, it is understood that “about” can modify each of the numbers in the series or range.

As used herein, concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Compositions or methods “comprising” or “including” one or more recited elements may include other elements not specifically recited. For example, a composition that “comprises” or “includes” a protein may contain the protein alone or in combination with other ingredients.

The singular forms of the articles “a,” “an,” and “the” include plural references unless the context clearly dictates otherwise. For example, the term “an antigen” or “at least one antigen” can include a plurality of antigens, including mixtures thereof.

Statistically significant means p≤0.05.

### II. Antibiotic Compounds

Derivatives of tolfenpyrad (tolfenpyrad derivatives) are described that have improved properties relative to tolfenpyrad. The tolfenpyrad derivatives have the formula represented by Formula I, or pharmaceutically acceptable salts or stereoisomers thereof:

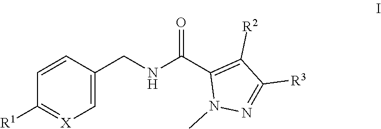

wherein:
X is CH or N;
R¹ is independently selected from the group consisting of C₁-C₁₀ alkyl, C₁-C₁₀ haloalkyl, C₃-C₁₀ cycloalkyl, C₁-C₁₀ alkoxy, 3-10 membered heterocycloalkyl, aryl, aryloxy, and heteroaryl;
R² and R³ are each independently selected from hydrogen, halogen, C₁-C₁₀ alkyl, C₂-C₁₀ alkenyl, C₂-C₁₀ alkynyl, C₁-C₁₀ haloalkyl, C₃-C₁₀ cycloalkyl, 4-10 membered heterocycloalkyl, aryl, aryloxy, and heteroaryl;
or,
R² and R³ are taken together with the carbons to which they are attached to form a C₃-C₁₀ cycloalkyl, 4-10 membered heterocycloalkyl, or heteroaryl; and,
R¹ cannot be

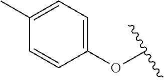 [SMILES: *Oc1ccc(C)cc1]

when R² is Cl and R³ is C₁-C₁₀ alkyl.

In some embodiments, R² cannot be Cl when R¹ is

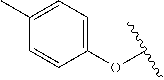 [SMILES: *Oc1ccc(C)cc1]

and R³ is C₁-C₁₀ alkyl. In some embodiments, R³ cannot be C₁-C₁₀ alkyl when R¹ is

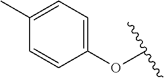 [SMILES: *Oc1ccc(C)cc1]

### and R² is Cl.

In some embodiments, R¹ is an aryl and X is CH.

In some embodiments, R¹ is selected from the group consisting of:

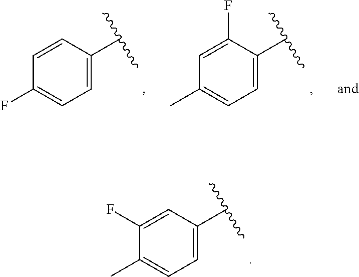

In some embodiments, R¹ is an aryloxy and X is CH.

In some embodiments, R¹ is

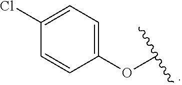 [SMILES: *Oc1ccc(Cl)cc1]

In some embodiments, R¹ is a heteroaryl.

In some embodiments, R¹ is a thiazole, a thiadiazole, or an oxadiazole. The thiadiazole can be 1,2,3-thiadiazole, 1,2,4-thiadiazole, 1,2,5-thiadiazole, or 1,3,4-thiadiazole. The oxadiazole can be 1,2,3-oxadiazole, 1,2,4-oxadiazole, 1,2,5-oxadiazole, or 1,3,4-oxadiazole.

In some embodiments, R¹ is a thiazole. In some embodiments, thiazole is selected from the group consisting of

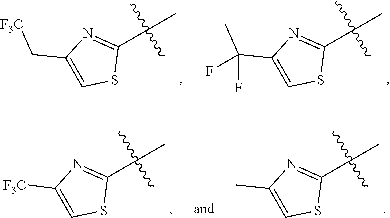

In some embodiments, R¹ is thiadiazole. In some embodiments, the thiadiazole is

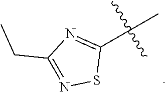 [SMILES: *c1nc(CC)ns1]

In some embodiments, R¹ is oxadiazole. In some embodiments, the oxadiazole is

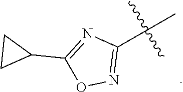 [SMILES: *c1noc(C2CC2)n1]

In some embodiments, R² is halogen. In some embodiments, the halogen is Cl.

In some embodiments, R² is hydrogen.

In some embodiments, R³ is C₁-C₁₀ alkyl. In some embodiments, the C₁-C₁₀ alkyl is a C₁-C₆ alkyl. In some embodiments, the C₁-C₆ alkyl is a C₁-C₂ alkyl. In some embodiments, the C₁-C₂ alkyl is an ethyl.

In some embodiments, R³ is hydrogen.

In some embodiments, R² and R³ together form a C₃-C₁₀ cycloalkyl. In some embodiments, the C₃-C₁₀ cycloalkyl is a cyclopentane.

The subject matter described herein includes, but is not limited to, the tolfenpyrad derivatives shown in Table 1.

TABLE 1
Tolfenpyrad derivatives identified in the F. novicida screening experiments.

| | |
|---|---|
| NK01 | 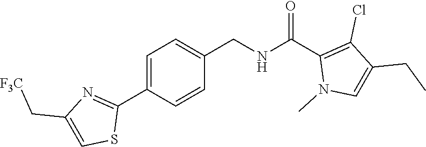 [SMILES: CCc1cn(C)c(C(=O)NCc2ccc(-c3nc(CC(F)(F)F)cs3)cc2)c1Cl] |
| NK02 | 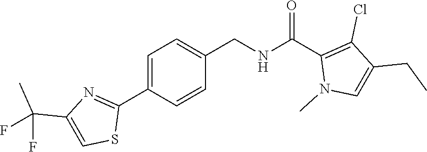 [SMILES: CCc1cn(C)c(C(=O)NCc2ccc(-c3nc(C(C)(F)F)cs3)cc2)c1Cl] |
| NK03 | 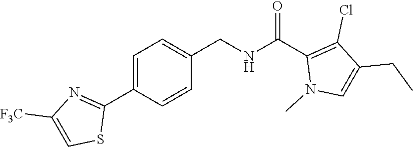 [SMILES: CCc1cn(C)c(C(=O)NCc2ccc(-c3nc(C(F)(F)F)cs3)cc2)c1Cl] |
| NK04 | 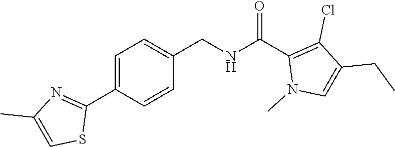 [SMILES: CCc1cn(C)c(C(=O)NCc2ccc(-c3nc(C)cs3)cc2)c1Cl] |
| NK05 | 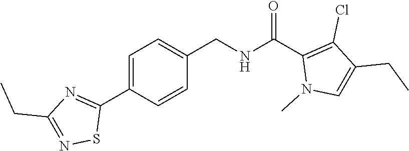 [SMILES: CCc1nsc(-c2ccc(CNC(=O)c3c(Cl)c(CC)cn3C)cc2)n1] |
| NK06 | 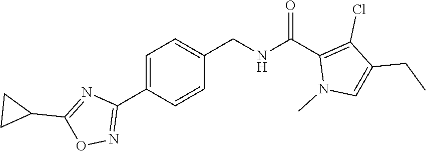 [SMILES: CCc1cn(C)c(C(=O)NCc2ccc(-c3noc(C4CC4)n3)cc2)c1Cl] |
| NK07 | 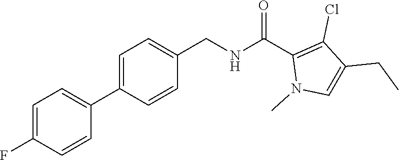 [SMILES: CCc1cn(C)c(C(=O)NCc2ccc(-c3ccc(F)cc3)cc2)c1Cl] |
| NK08 | 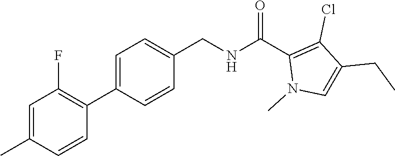 [SMILES: CCc1cn(C)c(C(=O)NCc2ccc(-c3ccc(C)cc3F)cc2)c1Cl] |
| NK09 | 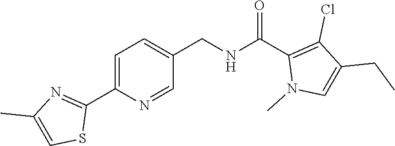 [SMILES: CCc1cn(C)c(C(=O)NCc2ccc(-c3nc(C)cs3)nc2)c1Cl] |
| NK10 | 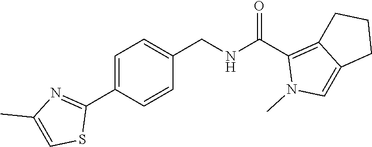 [SMILES: Cc1csc(-c2ccc(CNC(=O)c3c4c(cn3C)CCC4)cc2)n1] |
| NK11 | 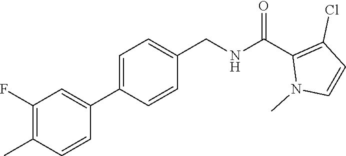 [SMILES: Cc1ccc(-c2ccc(CNC(=O)c3c(Cl)ccn3C)cc2)cc1F] |
| NK12 | 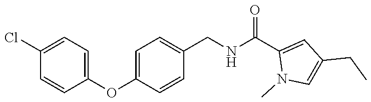 [SMILES: CCc1cc(C(=O)NCc2ccc(Oc3ccc(Cl)cc3)cc2)n(C)c1] |

or pharmaceutically acceptable salts or stereoisomers thereof.

### III. Pharmaceutical Compositions

Described are pharmaceutical compositions comprising at least one of the tolfenpyrad derivatives described herein, and one or more pharmaceutically acceptable excipients or carriers.

Pharmaceutically acceptable excipients (excipients) are substances other than an active pharmaceutical ingredient (API, therapeutic product) that are intentionally included with the API (molecule). Excipients do not exert or are not intended to exert a therapeutic effect at the intended dosage. Excipients may act to a) aid in processing of the API during manufacture, b) protect, support, or enhance stability, bioavailability or subject acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: adjuvants, absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The carrier can be, but is not limited to, a solvent or dispersion medium containing, for example, water, saline, phosphate buffered saline, Ringer's solution, ethanol, polyol (for example, glycerol, propylene glycol, and liquid polyethylene glycol), and suitable mixtures thereof. A carrier may also contain adjuvants or additives such as preservatives, wetting agents, emulsifying agents, and dispersing agents. A carrier may also contain isotonic agents, such as sugars, polyalcohols, sodium chloride, and the like.

For liquid formulations, for example, pharmaceutically acceptable carriers may be aqueous or non-aqueous solutions, suspensions, emulsions, or oils. Non-aqueous solvents include, for example, propylene glycol, polyethylene glycol, and injectable organic esters such as ethyl oleate. Aqueous carriers include, for example, water, alcoholic/aqueous solutions, emulsions or suspensions, including saline and buffered media. Examples of oils include those of petroleum, animal, vegetable, or synthetic origin, such as peanut oil, soybean oil, mineral oil, olive oil, sunflower oil, and fish-liver oil. Solid carriers/diluents include, for example, a gum, a starch (e.g., corn starch, pregeletanized starch), a sugar (e.g., lactose, mannitol, sucrose, or dextrose), a cellulosic material (e.g., microcrystalline cellulose), an acrylate (e.g., polymethylacrylate), calcium carbonate, magnesium oxide, talc, or mixtures thereof.

Optionally, sustained or directed release pharmaceutical compositions or vaccines can be formulated. This can be accomplished, for example, through use of liposomes or compositions wherein the active compound is protected with differentially degradable coatings (e.g., by microencapsulation, multiple coatings, and so forth). Such compositions may be formulated for immediate or slow release. It is also possible to freeze-dry the compositions and use the lyophilisates obtained (e.g., for the preparation of products for injection).

The pharmaceutical compositions can contain other additional components commonly found in pharmaceutical compositions. Such additional components can include, but are not limited to, anti-pruritics, astringents, local anesthetics, or anti-inflammatory agents (e.g., antihistamine, diphenhydramine, etc.).

### IV. Therapeutic Methods

In some embodiments, the subject matter described herein is directed to methods of treating disorders associated with Gram-negative intracellular bacterial pathogens, comprising administering a subject suffering from the disorder associated with the Gram-negative intracellular bacterial pathogen an effective amount of at least one tolfenpyrad derivative described herein or a pharmaceutically composition comprising at least one tolfenpyrad derivative described herein.

The described tolfenpyrad derivatives and pharmaceutical compositions comprising the described tolfenpyrad derivatives can be used to treat a Francisella species infection. In some embodiments, the Francisella species infection is a Francisella tularensis infection. In some embodiments, the Francisella tularensis infection is a Francisella tularensis subspecies tularensis (type A) infection or a Francisella tularensis subspecies holarctia (type B) infection.

Described are methods of treating a Francisella species infection comprising administering to a subject in need thereof, of an effective dose of any of the described tolfenpyrad derivatives or a pharmaceutical composition comprising any of the described tolfenpyrad derivatives. In some embodiments, the Francisella species infection is a Francisella tularensis infection. In some embodiments, the Francisella tularensis infection is a Francisella tularensis subspecies tularensis (type A) infection or a Francisella tularensis subspecies holarctia (type B) infection.

The described tolfenpyrad derivatives and pharmaceutical compositions comprising the described tolfenpyrad derivatives can be used to treat a disorder associated with a Francisella species. In some embodiments, the disorder associated with the Francisella species is a disorder associated with Francisella tularensis. In some embodiments, disorder associated with Francisella tularensis is a disorder associated with Francisella tularensis subspecies tularensis (type A) or a disorder associated with Francisella tularensis subspecies holarctia (type B). In some embodiments, the disorder is Tularemia.

Described are methods of treating a disorder associated with a Francisella species comprising administering to a subject in need thereof, of an effective dose of any of the described tolfenpyrad derivatives or pharmaceutical composition comprising any of the described tolfenpyrad derivatives. In some embodiments, the disorder associated with a Francisella species is a disorder associated with Francisella tularensis. In some embodiments, disorder associated with Francisella tularensis is a disorder associated with Francisella tularensis subspecies tularensis (type A) or a disorder associated with Francisella tularensis subspecies holarctia (type B). In some embodiments, the disorder is Tularemia.

In some embodiments, the method of treatment further comprises administering at least one additional active agent, such as an active agent useful in the treatment of disorders associated with intracellular bacterial pathogens. The additional compound(s) may optionally be administered concurrently. As used herein, the word “concurrently” means sufficiently close in time to produce a combined effect (that is, concurrently may be simultaneously, or it may be two or more events occurring within a short time period before or after each other).

In some embodiments, the subject is a mammal. The mammal can be, but is not limited to, a mouse, a rat, a rabbit, a dog, a cat, a livestock mammal (e.g., a cow, sheep or pig), a horse or a primate. The primate can be a human or a non-human primate. In some embodiments, the subject is a human. The subject may be of any age, including an infant, a juvenile, an adolescent, an adult, or a geriatric subject.

The therapeutically effective dosage of any specific compound can vary somewhat from compound to compound, and patient to patient, and will depend upon the condition of the patient and the route of delivery.

The subject matter is further described in the following non-limiting Examples. It should be understood that these Examples, while indicating preferred embodiments of the invention, are given by way of illustration only.

### EXAMPLES

### Example 1. Screening Tolfenpyrad Derivatives Reveals Compounds that Block Francisella Growth at Nanomolar Concentrations

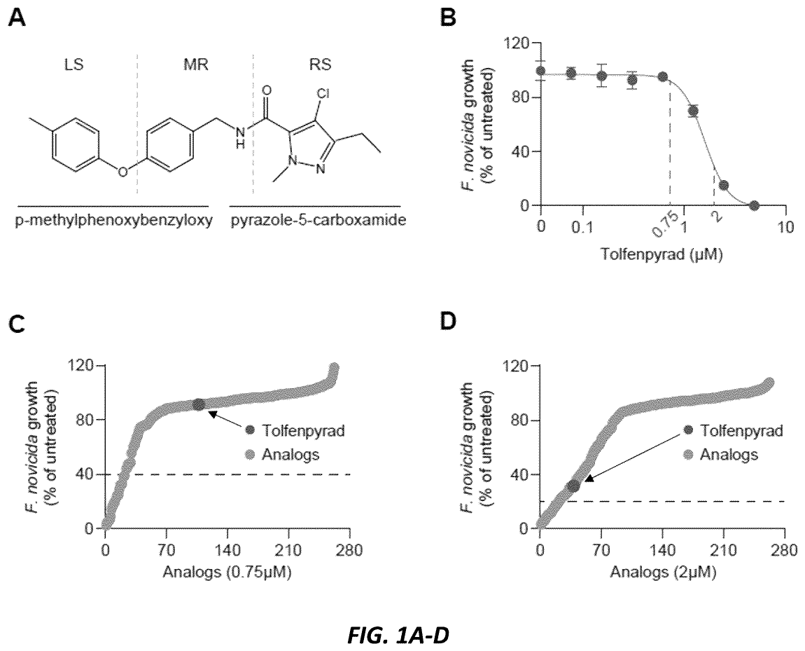

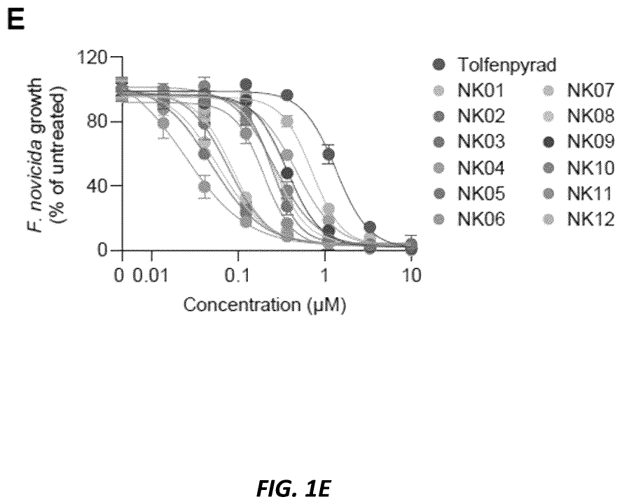

Tolfenpyrad is a structurally novel antibiotic that specifically inhibits the growth of species within the genus Francisella. Owing to a bulky, hydrophobic, and electron-rich p-methylphenoxybenzyloxy group, the physicochemical properties of tolfenpyrad stand to be improved ahead of further testing as a lead drug candidate. Structure-activity relationship studies were performed with the goal of improving these characteristics, while increasing antibacterial activity and decreasing toxicity to mammalian cells. To this end, a library of tolfenpyrad derivatives were screened. Derivatives of the tolfenpyrad scaffold were synthesized through the functionalization of pyrazole-5-carboxamide and p-methylphenoxybenzyloxy moieties by employing two established reaction schemes to derivatize the left side (LS) and right side (RS) of tolfenpyrad (FIG. 1A) as described in Le, T. G. et al. J Med Chem, 2019, 62, 1036-1053. The resultant set of 262 tolfenpyrad derivatives represented an array of physicochemical properties with substitutions on the LS, RS, and in the middle region (MR) of the tolfenpyrad scaffold.

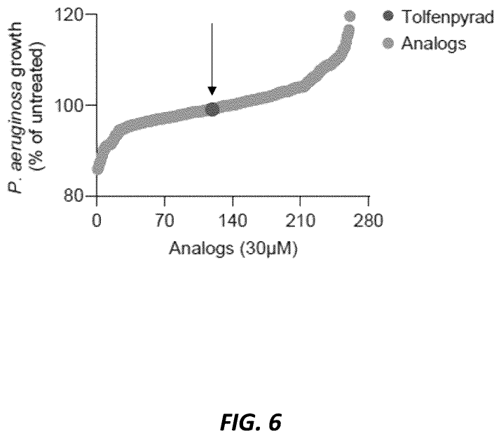

Tolfenpyrad's spectrum of activity is limited to species within Francisella, as comparison to strains in closely related genera are greater than 60-fold resistant. To establish whether the derivatives in this library have broader activity, they were screened for their ability to inhibit the growth of Pseudomonas aeruginosa at a sub-inhibitory concentration of 30 μM. Though some derivatives more potently inhibit P. aeruginosa growth than tolfenpyrad, even the most effective derivatives were only 10-15% more active (FIG. 6). This suggested that the bacterial pathway targeted by this class of compounds is unique to Francisella.

To investigate whether these derivatives exhibited increased activity against F. novicida compared with tolfenpyrad, dose-response assays were performed to determine optimum concentrations at which to detect more effective compounds. Tolfenpyrad blocks F. novicida growth by 10% and 70% (IC₁₀ and IC₇₀) at 0.75 μM and 2 μM, respectively (FIG. 1B). The derivatives were compared to tolfenpyrad for their ability to block the growth of F. novicida at these concentrations, the most potent ones were identified. In these experiments, 105 and 36 tolfenpyrad derivatives were more effective than tolfenpyrad at the IC₁₀ and IC₇₀, respectively (FIGS. 1C and 1D). An arbitrary threshold was set by focusing on derivatives that inhibited bacterial growth by ≥60% and 80% in the two screening experiments, and this yielded a total of 12 tolfenpyrad derivatives described herein (designated as NK01 to NK12) with substantially increased activity. The high-throughput nature of a screening experiment was essential to rapidly identifying active and inactive derivatives; however, a major limitation of this approach was the lack of a thorough quantitative measurement of the antibacterial activity. To accurately quantify the efficacy of these 12 tolfenpyrad derivatives, in vitro dose-response assays were performed on F. novicida growth, and the data obtained were subjected to non-linear regression analysis to calculate the half-maximal inhibitory concentrations (IC₅₀). Comparison of the IC₅₀ of tolfenpyrad to the tolfenpyrad derivatives revealed that the most potent compound, NK06, is ˜55-fold more active (FIG. 1E, Table 2, and Table 4). Additionally, several of these derivatives inhibited F. novicida growth at nanomolar concentrations.

TABLE 2
Calculated half maximal inhibitory concentrations (IC₅₀) and structures of the most
effective tolfenpyrad derivatives identified in the F. novicida screening experiments.

| | | | Activity compared | Site of |
|---|---|---|---|---|
| | | IC₅₀ | to Tolfenpyrad | structural |
| Compound | Structure | (μM) | (Fold increase) | modification |
| Tolfenpyrad | 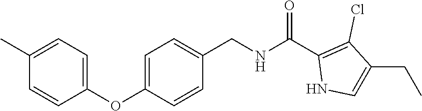 [SMILES: CCc1c[nH]c(C(=O)NCc2ccc(Oc3ccc(C)cc3)cc2)c1Cl] | 1.4 | — | — |
| NK01 | 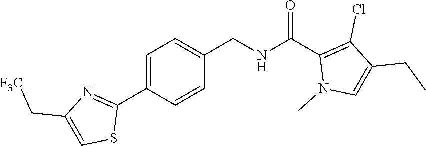 [SMILES: CCc1cn(C)c(C(=O)NCc2ccc(-c3nc(CC(F)(F)F)cs3)cc2)c1Cl] | 0.06 | 22.6 | LS |
| NK02 | 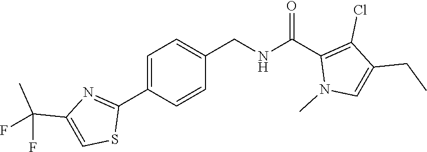 [SMILES: CCc1cn(C)c(C(=O)NCc2ccc(-c3nc(C(C)(F)F)cs3)cc2)c1Cl] | 0.08 | 17.6 | LS |
| NK03 | 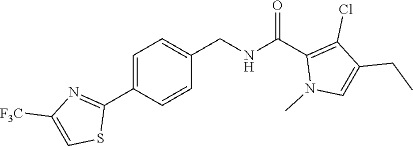 [SMILES: CCc1cn(C)c(C(=O)NCc2ccc(-c3nc(C(F)(F)F)cs3)cc2)c1Cl] | 0.25 | 5.5 | LS |
| NK04 | 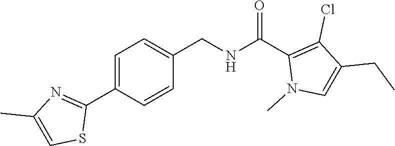 [SMILES: CCc1cn(C)c(C(=O)NCc2ccc(-c3nc(C)cs3)cc2)c1Cl] | 0.48 | 2.9 | LS |
| NK05 | 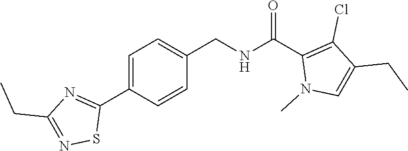 [SMILES: CCc1nsc(-c2ccc(CNC(=O)c3c(Cl)c(CC)cn3C)cc2)n1] | 0.05 | 27.4 | LS |
| NK06 | 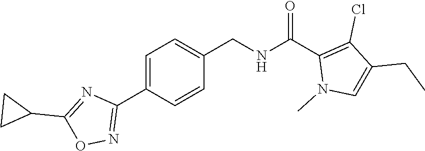 [SMILES: CCc1cn(C)c(C(=O)NCc2ccc(-c3noc(C4CC4)n3)cc2)c1Cl] | 0.02 | 55.1 | LS |
| NK07 | 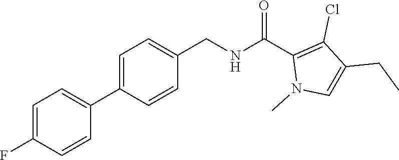 [SMILES: CCc1cn(C)c(C(=O)NCc2ccc(-c3ccc(F)cc3)cc2)c1Cl] | 0.09 | 15.7 | LS |
| NK08 | 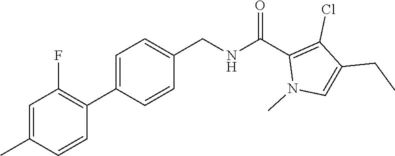 [SMILES: CCc1cn(C)c(C(=O)NCc2ccc(-c3ccc(C)cc3F)cc2)c1Cl] | 0.26 | 5.2 | LS |
| NK09 | 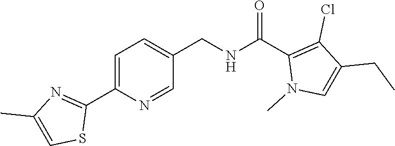 [SMILES: CCc1cn(C)c(C(=O)NCc2ccc(-c3nc(C)cs3)nc2)c1Cl] | 0.37 | 3.7 | LS + MR |
| NK10 | 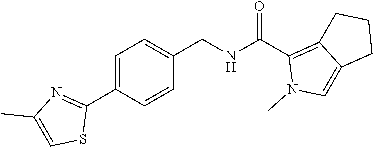 [SMILES: Cc1csc(-c2ccc(CNC(=O)c3c4c(cn3C)CCC4)cc2)n1] | 0.27 | 5.1 | LS + RS |
| NK11 | 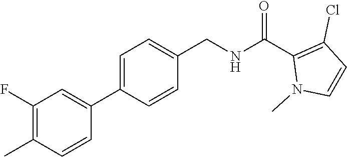 [SMILES: Cc1ccc(-c2ccc(CNC(=O)c3c(Cl)ccn3C)cc2)cc1F] | 0.20 | 6.9 | LS + RS |
| NK12 | 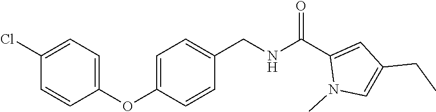 [SMILES: CCc1cc(C(=O)NCc2ccc(Oc3ccc(Cl)cc3)cc2)n(C)c1] | 0.72 | 1.9 | LS + RS |

Scrutinizing the structural changes from tolfenpyrad in these 12 derivatives revealed specific modifications that correlated with higher activity. Eight compounds contained changes to the LS, one was modified on both the LS and MR, and three have modifications to the LS and RS (Table 2). Notably, the tolyloxy group on the LS was altered in all of these 12 derivatives. Of the tolfenpyrad derivatives with modifications exclusively to the LS, six have substitutions to alkylated or fluoroalkylated azoles. Four of these azole substitutions are thiazoles (NK01-NK04), one a thiadiazole (NK05), and another an oxadiazole (NK06). These six derivatives have various alkyl or fluoro alkyl groups on the azoles. The two remaining derivatives with substitutions on the LS have the tolyloxy group substituted for fluorobenzene and fluorotoluene (NK07 and NK08, respectively). NK09 has a modification on both the LS and MR, and is very similar to NK04, with the only difference being a substitution of the central benzene ring for pyridine. Of the derivatives that have LS and RS modifications, the LS substitutions are similar to others in this subset. For example, NK10 shares an identical LS substitution with NK04 and NK09. Conversely, within the group of LS and RS-modified compounds, the RS modifications were unique for this subset. These substitutions included the introduction of a five-membered ring to NK10 or deletion of the ethyl- or chloro-groups from the pyrazole ring in NK11 and NK12, respectively. These conservative modifications to tolfenpyrad led to a significant increase in activity; however, the degree to which these changes increased the bacterial targeting activity is unclear.

F. novicida is a representative model for Francisella species and shares up to 97% nucleotide identity with mammalian pathogens in the genus. Human-pathogenic Francisella species are categorized as either type A or type B, which cause an acute or more mild disease, respectively. To determine whether the three tolfenpyrad derivatives that were most potent against F. novicida displayed increased activity on type A and B strains, the broth microdilution method set by the Clinical and Laboratory Standards Institute (CLSI) was employed on NK01, NK05, and NK06 at biosafety level-3 (Weyermann, J., Lochmann, D. & Zimmer, A. International Journal of Pharmaceutics 2005, 288, 369-376). Tolfenpyrad was effective against all strains of Francisella, but lacked activity against Pseudomonas aeruginosa, Escherichia coli, and Staphylococcus aureus (Table 3). Conversely, the broad-spectrum antibiotic Ciprofloxacin was effective against all bacterial strains tested. Similar to tolfenpyrad, NK01, NK05, and NK06 were active against the biosafety level-3 strains of Francisella tested but lacked activity on the other Gram-negative and Gram-positive bacteria. Compared to tolfenpyrad, these derivatives displayed improved activity on the human-virulent strains of Francisella, in some cases increasing activity >200-fold. Depending on the Francisella strain tested, the activities of these derivatives were up to 9-fold more effective than Ciprofloxacin, which is the clinical standard treatment against tularemia in humans. The in vitro activity of an antibacterial compound is just one of several important characteristics for consideration. A second important property of an antibacterial that is intended for use on, or in, a host, is low toxicity.

TABLE 3
Minimal inhibitory concentrations of ciprofloxacin, tolfenpyrad, and the three most
effective tolfenpyrad derivatives identified in the screening experiments on human-virulent
strains of Francisella, P. aeruginosa, E. coli and S. aureus.

| | | | | | |
|---|---|---|---|---|---|
| Species | | | | | |
| and strain | Ciprofloxacin | Tolfenpyrad | NK01 | NK05 | NK06 |

F. tularensis subsp. tularensis (Type A)

| | | | | | |
|---|---|---|---|---|---|
| NIH B-38 | ≤24 nM | 326 nM | 34 nM | ≤3 nM | 10 nM |
| | (≤0.008 μg/mL) | (0.125 μg/mL) | (0.015 μg/mL) | (0.001 μg/mL) | (0.004 μg/mL) |
| MA00-2987 | 91 nM | 1303 nM | 34 nM | 10 nM | 155 nM |
| | (0.03 μg/mL) | (0.5 μg/mL) | (0.015 μg/mL) | (0.004 μg/mL) | (0.06 μg/mL) |
| WY96-3418 | 45 nM | 2605 nM | 135 nM | 10 nM | 648 nM |
| | (0.015 μg/mL) | (1 μg/mL) | (0.06 μg/mL) | (0.004 μg/mL) | (0.25 μg/mL) |
| SchuS4 | ≤24 nM | 2605 nM | 135 nM | 77 nM | 648 nM |
| | (≤0.008 μg/mL) | (1 μg/mL) | (0.06 μg/mL) | (0.03 μg/mL) | (0.25 μg/mL) |

F. tularensis subsp. holarctica (Type B)

| | | | | | |
|---|---|---|---|---|---|
| KY99-3387 | 91 nM | 651 nM | 34 nM | ≤3 nM | 155 nM |
| | (0.03 μg/mL) | (0.25 μg/mL) | (0.015 μg/mL) | (0.001 μg/mL) | (0.06 μg/mL) |
| HN63 | 45 nM | 1303 nM | 68 nM | 21 nM | 155 nM |
| | (0.015 μg/mL) | (0.5 μg/mL) | (0.03 μg/mL) | (0.008 μg/mL) | (0.06 μg/mL) |
| OR96-0246 | 45 nM | 1303 nM | 68 nM | 38 nM | 648 nM |
| | (0.015 μg/mL) | (0.5 μg/mL) | (0.03 μg/mL) | (0.015 μg/mL) | (0.25 μg/mL) |
| LVSR | ≤24 nM | 313 nM | 34 nM | 5 nM | 21 nM |
| | (≤0.008 μg/mL) | (0.12 μg/mL) | (0.015 μg/mL) | (0.002 μg/mL) | (0.008 μg/mL) |

P. aeruginosa

| | | | | | |
|---|---|---|---|---|---|
| Boston | 0.36 μM | >5 μM | >5 μM | >5 μM | >5 μM |
| 41501 | (0.12 μg/mL) | (>2 μg/mL) | (>2 μg/mL) | (>2 μg/mL) | (>2 μg/mL) |
| DSM 1103 | ≤0.02 μM | >5 μM | >5 μM | >5 μM | >5 μM |
| | (≤0.008 μg/mL) | (>2 μg/mL) | (>2 μg/mL) | (>2 μg/mL) | (>2 μg/mL) |
| Wichita | 0.18 μM | >5 μM | >5 μM | >5 μM | >5 μM |
| | (0.06 μg/mL) | (>2 μg/mL) | (>2 μg/mL) | (>2 μg/mL) | (>2 μg/mL) |

### Example 2. Evaluation for Toxicity Reveals Three Tolfenpyrad Derivatives that are Less Toxic and More Effective than Tolfenpyrad

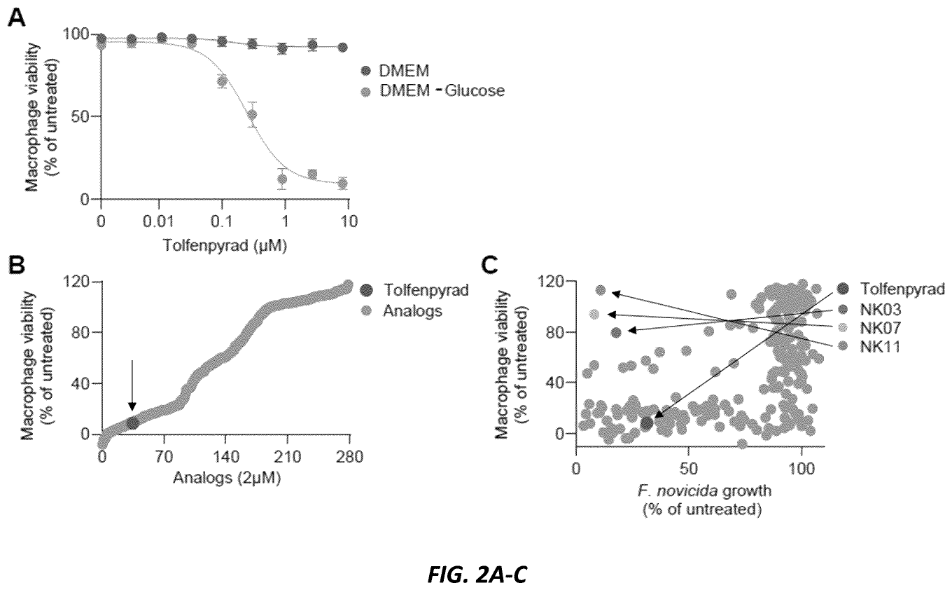

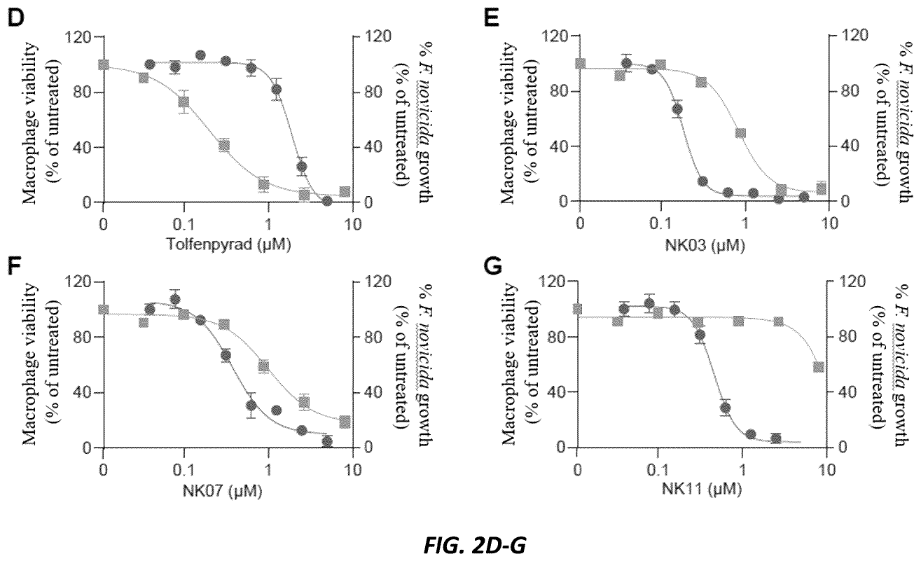

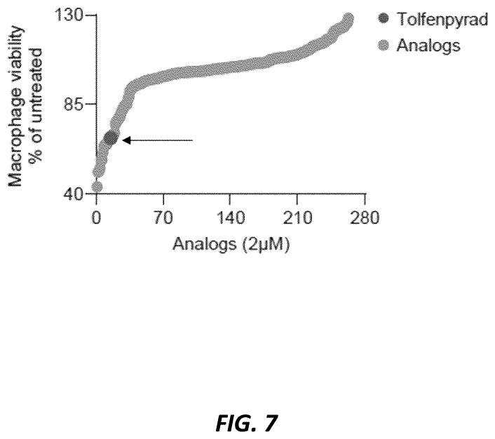

In vitro cytotoxicity assays using cultured cells can provide preliminary insight into cellular toxicity and are a prerequisite for in vivo studies. However, these assays can be prone to false negative results due to either an oversimplified tissue culture model or the absence of the conditions necessary to demonstrate toxicity. This is particularly the case for drugs that inhibit mitochondrial function by disrupting the electron transport chain to induce oxidative stress. The established mechanism of toxicity for tolfenpyrad in metazoan cells is through inhibiting mitochondrial metabolism; therefore, elucidating the mitochondrial toxicity of the library of derivatives was an important step toward finding a derivative that lacked acute toxicity. Rapidly dividing cells in vitro generate most ATP via glycolysis, despite abundant oxygen and functional mitochondria, a circumstance known as the Crabtree effect. Based on this information, it was determined that the primary in vitro model for Francisella infection, macrophages cultured in Dulbecco's Modified Eagle Medium (DMEM), may be a poor assay for acute toxicity for this class of compounds. To determine if immortalized bone marrow-derived murine macrophages (iBMDM) display increased sensitivity to tolfenpyrad in the absence of glycolysis, they were incubated in a titration of tolfenpyrad in DMEM or a DMEM formulation where glucose is replaced with galactose (DMEM glucose) and measured cell viability. iBMDM cells were viable in the presence of glucose (DMEM) regardless of the concentration of tolfenpyrad. Conversely, in glucose-free DMEM, tolfenpyrad induced iBMDM death in a dose-dependent manner with almost complete loss of viability below 1 μM. These data suggested that culturing iBMDM cells with drugs in DMEM was a poor assay for finding and excluding mitochondrial toxins (FIG. 2A). Consistent with this, there was very little toxicity observed when iBMDM cells were incubated with the tolfenpyrad derivatives in the presence of glucose (FIG. 7).

Incubation of iBMDM cells with drugs in the absence of glucose represents a highly sensitive assay to query for mitochondrial toxins in macrophages. To determine if the tolfenpyrad derivatives were mitochondrial toxins in cultured macrophages, iBMDM cells in glucose-free DMEM were treated with 2 μM of derivatives for 16 h and quantified cell viability. In these conditions, approximately three quarters of the derivatives were less toxic than tolfenpyrad and 40% of them lacked acute toxicity (FIG. 2B). To directly compare each derivative's in vitro toxicity with its ability to inhibit the growth of F. novicida, iBMDM toxicity against F. novicida growth inhibition was plotted (FIG. 2C). This analysis revealed that NK03, NK07, and NK11 have significantly improved efficacy against F. novicida while harboring little to no toxicity (Table 4).

TABLE 4
Antibacterial-activity and cytotoxicity of tolfenpyrad and the 12 most effective
tolfenpyrad derivatives identified in the screening experiments.

| | F. novicida growth | | | | Mouse macrophage viability | |
|---|---|---|---|---|---|---|
| | | | T-test | P. | 2 μM | T-test |
| | 0.75 μM | 2 μM | comparing 2 | aeruginosa | analogs (% | comparing 2 |
| | analogs | analogs | μM analogs to | growth | of untreated | μM analogs to |
| | (% of | (% of | tolfenpyrad | (30 μM, % | in, DMEM- | tolfenpyrad |
| Compound | control) | control) | control | of control) | glucose) | control |
| Tolfenpyrad | 91.1 | 31.4 | 1.00 | 89.0 | 7.8 | 5.99E−01 |
| NK01 | 2.3 | 3.2 | 3.45E−19 | 93.3 | 6.2 | 8.18E−01 |
| NK02 | 4.4 | 5.7 | 2.95E−16 | 95.3 | 23.2 | 2.13E−02 |
| NK03 | 19.8 | 17.8 | 1.04E−07 | 96.9 | 79.5 | 1.82E−02 |
| NK04 | 32.8 | 12.0 | 2.44E−12 | 96.7 | 0.7 | 3.28E−02 |
| NK05 | 18.9 | 11.0 | 5.75E−13 | 97.5 | 22.3 | 3.12E−02 |
| NK06 | 17.4 | 11.6 | 6.41E−13 | 96.1 | 20.7 | 2.21E−02 |
| NK07 | 4.9 | 8.1 | 1.40E−15 | 96.9 | 94.0 | 7.31E−07 |
| NK08 | 6.5 | 5.0 | 4.01E−16 | 99.2 | 47.4 | 1.60E−04 |
| NK09 | 20.0 | 8.0 | 1.21E−15 | 97.1 | 53.9 | 1.68E−03 |
| NK10 | 39.3 | 6.1 | 3.18E−09 | 96.4 | 8.0 | 9.70E−01 |
| NK11 | 39.0 | 10.9 | 1.84E−13 | 99.7 | 113.0 | 4.49E−05 |
| NK12 | 33.4 | 9.4 | 3.56E−13 | 98.5 | 16.2 | 5.91E−02 |

To describe the relationship between bacterial growth inhibition with toxicity, these derivatives were titrated onto iBMDM cells in DMEM lacking glucose and measured mammalian cell viability. In a separate assay, F. novicida growth was measured by monitoring OD₆₀₀ in the presence of the derivatives, and these results were plotted together to show the therapeutic window of tolfenpyrad, NK03, NK07, and NK11 (FIGS. 2D, 2E, 2F, and 2G). In these conditions, tolfenpyrad only affected bacterial growth at a concentration where it has greater than 80% toxicity to mammalian cells (FIG. 2D). Conversely, NK03, NK07, and NK11 displayed antibacterial activity at concentrations lower than those that displayed toxicity toward mammalian cells. Notably, NK11 had an undefined therapeutic window with maximal antibacterial activity at a concentration that did not result in observed toxicity in macrophages.

### Example 3. Non-Toxic Tolfenpyrad Derivatives Block Intramacrophage Growth of F. novicida and Pathogenicity In Vivo

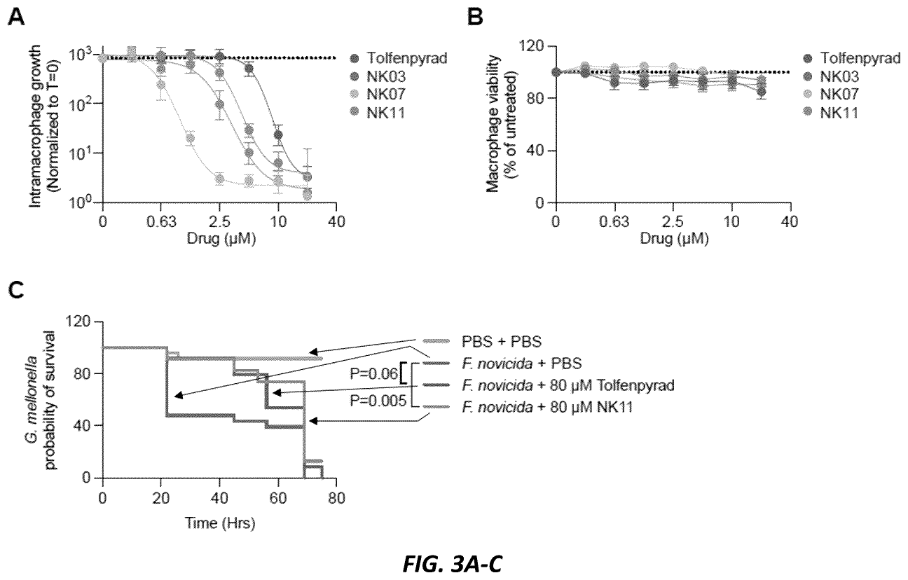

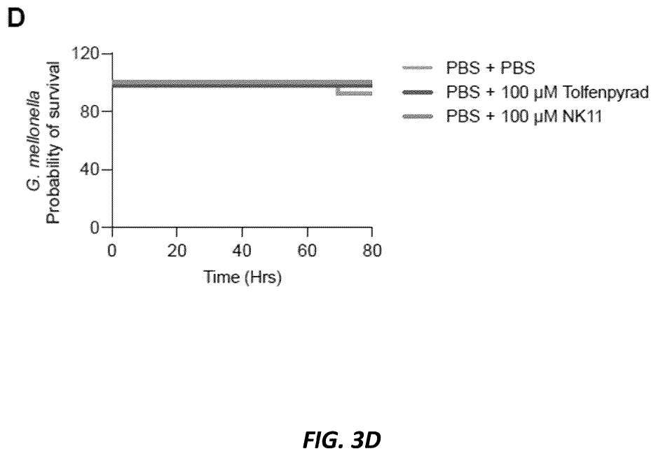

The mammalian cell membrane represents a formidable barrier that can exclude many drugs from intracellular targets. Tolfenpyrad inhibits cellular respiration in insects, and it can transverse the mammalian cell membrane to exert control of F. novicida growth within iBMDM cells. One potential mechanism by which NK03, NK07, and NK11 displayed reduced toxicity to mammalian cells is that the changes in their structures prevent their translocation into iBMDM cells. To test if these derivatives transversed the host cell membrane and blocked intracellular growth of F. novicida, a modified gentamicin protection assay was employed. iBMDM cells were briefly incubated with bacteria in DMEM to allow for phagocytosis, followed by clearance of extracellular bacteria with gentamicin treatment. After extensive washing, cells were treated with a titration of NK03, NK07, NK11, and tolfenpyrad, and intracellular bacteria were enumerated immediately and 16 h post infection. Tolfenpyrad and all derivatives inhibited intracellular F. novicida growth in a dose-dependent manner and did not affect macrophage viability in the absence of bacteria (FIGS. 3A and 3B). Tolfenpyrad was least effective at inhibiting bacterial growth with an IC₅₀ of 7.38 μM (Table 5). NK03 and NK11 were more active than tolfenpyrad, with IC₅₀ values 2-3-fold lower than tolfenpyrad, and NK07 inhibited bacterial growth about 10-fold more effectively than tolfenpyrad with an IC₅₀ of 0.77 μM. These data suggested that the less toxic derivatives translocated into mammalian cells with equal efficiency as tolfenpyrad or their reduced translocation is offset by increased activity.

TABLE 5
IC₅₀ values of tolfenpyrad and the three non-toxic and more effective
tolfenpyrad derivatives on intramacrophage growth of F. novicida.

| Compound | IC₅₀ (μM) |
|---|---|
| Tolfenpyrad | 7.38 |
| NK03 | 3.53 |
| NK07 | 0.77 |
| NK11 | 2.56 |

The larval stage of Galleria mellonella have been used extensively as an invertebrate model for bacterial infection, and its popularity is increasing as an in vivo model for tularemia. G. mellonella have been employed successfully to quantify both the virulence of different strains and subspecies of Francisella and the efficacy of antimicrobial agents. Since NK11 was the least toxic of the derivatives and displayed improved anti-Francisella activity, G. mellonella was employed to determine if it is effective in vivo. Larvae were injected in the hemocoel with 10 μL PBS containing 10⁵ CFU of F. novicida and subsequently injected with 10 μL of 80 μM tolfenpyrad or NK11. Injection of F. novicida resulted in 50% death after one day and 100% death by three days post infection (FIG. 3C). Treatment with either tolfenpyrad or NK11 delayed death, with NK11 slightly more effective than tolfenpyrad. By 69 hours, most of the larvae injected with bacteria died, regardless of tolfenpyrad or derivative intervention. Groups that were left uninjected, those injected with two successive doses of PBS, and those injected with PBS followed by 100 μM tolfenpyrad or NK11 all displayed less than 10% death for the duration of the experiment (FIG. 3D and data not shown). The lack of tolfenpyrad-induced toxicity at this concentration indicated that NK11 was not toxic to G. mellonella.

Although the most effective tolfenpyrad derivatives (NK01, NK05 and NK06) were more potent in vitro, they displayed significant levels of toxicity to macrophages in conditions that require aerobic respiration. NK03, NK07 and NK11 were less active in vitro, but they may represent more promising compounds from a drug development standpoint because they were less toxic and were able to attenuate the intracellular growth and pathogenicity of F. novicida in vivo. To determine if these least toxic derivatives were effective at attenuating type A and B strains, the broth microdilution method described above was used. These derivatives were ineffective toward P. aeruginosa, E. coli, or S. aureus; however, they were active against both type A and type B strains of Francisella, albeit at greater concentrations than required by the most active tolfenpyrad derivatives (Table 6).

TABLE 6
Minimal inhibitory concentrations (MIC) of three non-toxic and more effective
tolfenpyrad derivatives identified in the screening experiments on strains of Francisella,
P. aeruginosa, E. coli and S. aureus.

Species

| | | | |
|---|---|---|---|
| and strain | NK03 | NK07 | NK11 |

F. tularensis subsp. tularensis (Type A)

| | | | | | | |
|---|---|---|---|---|---|---|
| NIH B-38 | 141 nM | (0.06 μg/mL) | 11 nM | (0.004 μg/mL) | 42 nM | (0.015 μg/mL) |
| MA00-2987 | 588 nM | (0.25 μg/mL) | 81 nM | (0.03 μg/mL) | 335 nM | (0.12 μg/mL) |
| WY96-3418 | >4707 nM | (>2 μg/mL) | 81 nM | (0.03 μg/mL) | 335 nM | (0.12 μg/mL) |
| SchuS4 | 2353 nM | (1 μg/mL) | 161 nM | (0.06 μg/mL) | 699 nM | (0.25 μg/mL) |

F. tularensis subsp. holarctica (Type B)

| | | | | | | |
|---|---|---|---|---|---|---|
| KY99-3387 | 282 nM | (0.12 μg/mL) | 40 nM | (0.015 μg/mL) | 335 nM | (0.12 μg/mL) |
| HN63 | 588 nM | (0.25 μg/mL) | 81 nM | (0.03 μg/mL) | 699 nM | (0.25 μg/mL) |
| OR96-0246 | 1177 nM | (0.5 μg/mL) | 81 nM | (0.03 μg/mL) | 699 nM | (0.25 μg/mL) |
| LVSR | 70 nM | (0.03 μg/mL) | 40 nM | (0.015 μg/mL) | 42 nM | (0.015 μg/mL) |

P. aeruginosa

| | | | | | | |
|---|---|---|---|---|---|---|
| Boston 41501 | >5 μM | (>2 μg/mL) | >5 μM | (>2 μg/mL) | >5 μM | (>2 μg/mL) |
| DSM 1103 | >5 μM | (>2 μg/mL) | >5 μM | (>2 μg/mL) | >5 μM | (>2 μg/mL) |
| Wichita | >5 μM | (>2 μg/mL) | >5 μM | (>2 μg/mL) | >5 μM | (>2 μg/mL) |

### Example 4. Tolfenpyrad and Tolfenpyrad Derivatives Share a Common Mechanism of Action

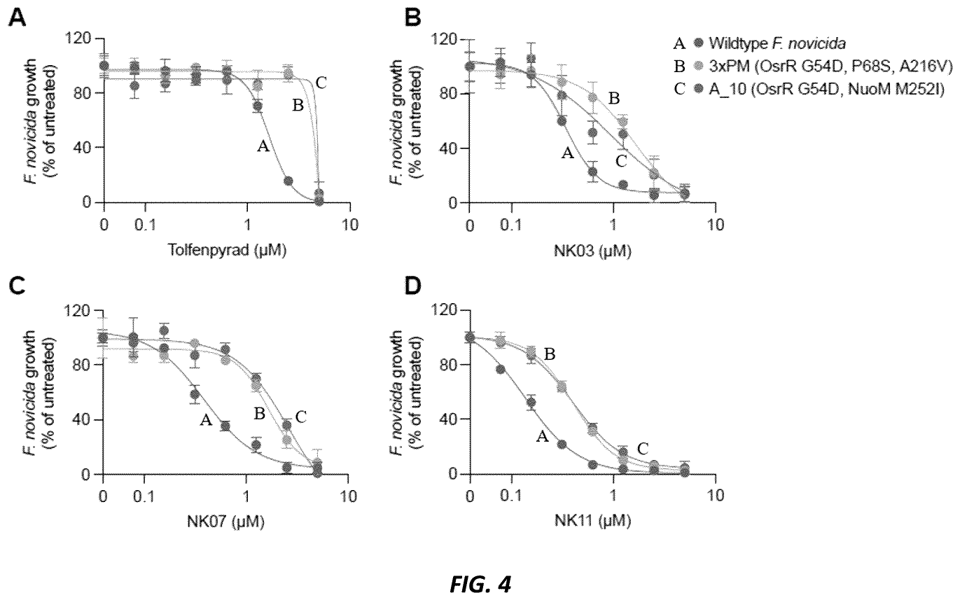

Compared to tolfenpyrad, the tested derivatives significantly inhibited F. novicida pathogenicity and blocked the growth of type A and type B strains of F. tularensis. The genetic basis of resistance of isolated tolfenpyrad-resistant strains of F. novicida were traced to mutations in two genes, osrR and nuoM. OsrR is a poorly characterized transcriptional regulator, and mutation of three amino acids (G54D, P68S, A216V) generates a strain (3×PM) that is approximately 5-fold resistant to tolfenpyrad (FIG. 4A, Table 7). NuoM is a component of complex I in the respiratory electron transport chain and is essential in Francisella. A tolfenpyrad-selected isolate, A_10, is resistant to tolfenpyrad and contains mutations in both OsrR (G54D) and NuoM (M252I). Experiments were conducted to explore if tolfenpyrad and the more active derivatives were likely to target the same pathways by quantifying the sensitivity of these mutants to the derivatives. Dose response assays and calculation of the IC₅₀ values revealed that these mutations yield similar levels of resistance to the derivatives as they do to tolfenpyrad (FIG. 4B-4D, Table 7). Therefore, the genetic mutations in F. novicida that are associated with the loss of sensitivity to tolfenpyrad similarly affected the derivatives, suggesting that these derivatives may target the same bacterial pathway(s).

TABLE 7
Calculated IC₅₀ values of tolfenpyrad and three
non-toxic and more effective tolfenpyrad derivatives
on wildtype F. novicida, 3xPM (OsrR G54D, P68S, A216V),
and A_10 (OsrR G54D, NuoM M252I).

| | | 3xPM (OsrR G54D, | | A_10 (OsrR G54D, | |
|---|---|---|---|---|---|
| | Wildtype | P68S, A216V) | | NuoM M252I | |
| | IC₅₀ | IC₅₀ | Fold increase | IC₅₀ | Fold increase |
| Compound | (μM) | (μM) | from WT | (μM) | from WT |
| Tolfenpyrad | 1.63 | 7.84 | 4.8 | 6.16 | 3.8 |
| NK03 | 0.34 | 1.64 | 4.8 | 0.94 | 2.8 |
| NK07 | 0.39 | 1.65 | 4.3 | 2.67 | 6.8 |
| NK11 | 0.14 | 0.40 | 2.9 | 0.40 | 2.9 |

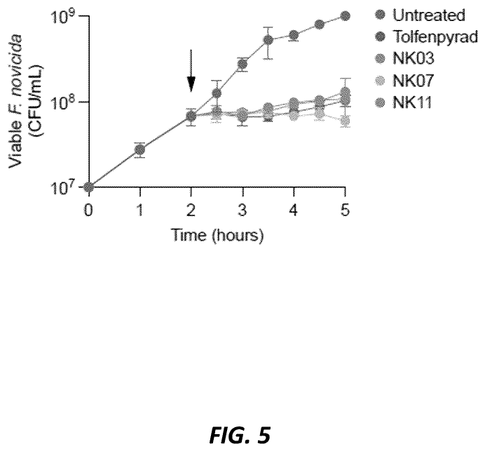

Tolfenpyrad inhibited the growth of F. novicida by inducing bacteriostasis. To better understand the mechanism that the tolfenpyrad derivatives exert to attenuate Francisella, these derivatives were introduced into cultures of actively growing F. novicida and assessed their effect on bacterial viability. Viable bacteria were enumerated before and after the addition of the derivatives to establish whether the derivatives were more effective through a change in activity from bacteriostatic to bactericidal. Similar to tolfenpyrad, NK03, NK07, and NK11 exhibited a bacteriostatic activity, with no loss of bacterial viability within 3 hours of the addition (FIG. 5). These results showed that modifications to the tolfenpyrad scaffold increased activity without changing the targeted bacterial pathways.

### Example 5. Materials and Methods

A. Bacterial strains and culture conditions: F. novicida Utah 112 (U112, NR-13), F. tularensis Schu S4 (NR-643), F. tularensis MA00-2987 (NR-645), F. tularensis WY96-3418 (NR-644), F. tularensis HN63 (NR-36146), F. tularensis NIH B-38 (NR-50), F. tularensis 100 (NR-36142), F. holarctica LVSR (capsule-negative variant, NR597), F. holarctica CDC-LVS (NR-646), F. holarctica KY99-3387 (NR-647), and F. holarctica OR96-0246 (NR-648) were obtained from the Biodefense and Emerging Infections Research Resources Repository (BEI resources) at the National Institute of Allergy and Infectious Diseases (NIAID) at the National Institutes of Health (NIH). P. aeruginosa Boston 41501 (ATCC 27853), S. aureus Wichita (ATCC 29213), and E. coli DSM 1103 (ATCC 25922) were obtained from the American Type Culture Collection. Bacteria were cultured in tryptic soy broth supplemented with 0.1% cysteine (TSBC), except in experiments to determine the minimum inhibitory concentrations, which cultured bacteria in cation-adjusted Mueller-Hinton broth (CAMHB) containing 2% IsoVitaleX.

B. Screening tolfenpyrad analogs for antibacterial activity: Tolfenpyrad derivatives were synthesized at the Monash Institute of Pharmaceutical Sciences, Monash University, Australia. These derivatives were then prepared to a 20 mM concentration in 100% DMSO, and their physicochemical properties were calculated by using Swiss ADME (Le, T. G. et al. J Med Chem 2019, 62, 1036-1053 and Eshraghi, A. et al. Cell Host & Microbe 2016, 20, 573-583). F. novicida and P. aeruginosa were grown until they reached an optical density at 600 nm (OD_{600 nm}) of approximately 1.0, then diluted to OD_{600 nm}=0.05. The derivatives were diluted in bacteriological media to twice the intended final concentration and mixed in equal volumes with diluted bacteria in 384-well plates. The plates were covered with gas-permeable seals and incubated overnight at 37° C. in a shaking plate incubator, while OD_{600 nm} was measured kinetically every 20 minutes. The OD_{600 nm} of the derivative-treated bacteria was analyzed at a timepoint when the untreated controls were in the late exponential growth phase (set to 100%). All screening experiments were performed with four technical replicates and a single biological replicate due to limited compound availability; however, the F. novicida screen was performed at two different drug concentrations to increase confidence in the results. Compounds identified as growth inhibitory in the primary screens were picked from the library and the results were validated by repeating the assays at multiple concentrations as described in the text.

C. Dose-response curves and measurement of half-maximal inhibitory concentration (IC50): Derivatives were prepared in serial dilutions to two times the indicated concentrations in 0.1% DMSO and transferred to 384-well plates. F. novicida was grown to mid-log phase, diluted to OD_{600 nm}=0.05, then dispensed into the plates with equal volume as the diluted derivatives. The plates were incubated at 37° C. with continuous shaking in a plate reader while OD_{600 nm} was measured every 20 minutes. To calculate the IC₅₀, the OD_{600 nm} of all the derivative-treated groups was recorded when the untreated group reached late log phase. Untreated controls were set to 100% to graph the dose response curves, and a variable-slope, four-parameter nonlinear regression model was employed to calculate the IC₅₀ values. All experiments were performed with three to six technical replicates and at least three biological replicates.

D. Determining minimum inhibitory concentrations: The broth microdilution technique was employed as described by the Clinical and Laboratory Standards Institute (CLSI) to determine minimum inhibitory concentrations (Heine, H. S., Miller, L., Halasohoris, S. & Purcell, B. K. Antimicrob Agents Chemother 2017, 61). Tolfenpyrad and derivatives were serially diluted in two-fold steps in CAMHB and 50 μL of each was added to 96-well plates. Inocula were prepared in CAMHB containing 4% Isovitalex and added to the 96-well plates in an equal volume to achieve a final bacterial cell density of 5×10⁴ CFU/well and Isovitalex concentration of 2%. Plates were incubated at 37° C. and MICs were determined visually at 24-48 hours.

E. Mammalian cell culture and evaluation of drug-induced cytotoxicity: Immortalized Mus musculus bone marrow derived macrophages (iBMDMs, NR-9456) were obtained from the BEI resources, NIAID, NIH, and maintained at 37° C. with 5% CO₂ in DMEM supplemented with 10% fetal bovine serum, 4.5 g/L glucose, 2 mM glutamine, 110 mg/L sodium pyruvate, 100 U/mL penicillin, and 100 μg/mL streptomycin. 4.5×10⁴ iBMDM cells were seeded into 96-well plates and incubated overnight in the absence of antibiotics. The subsequent day, derivatives were diluted in the tissue culture media to three times the indicated concentrations, added to the cells at a ratio of one to three, and incubated for 16 hours. Following incubation, a cell viability assay was performed according to the manufacturer's instructions (Promega nonradioactive cytoxicity assay). Screening experiments were performed with four technical replicates and a single biological replicate due to limited compound availability. Derivatives of interest in the primary screen were picked from the library and the results were validated by repeating the assays for measurement of dose response to multiple concentrations. For dose response assays, all experiments were performed with three to six technical replicates and at least three biological replicates.

F. Intramacrophage F. novicida growth assay: iBMDM infections were performed as previously described (Eshraghi, A. et al. Cell Host & Microbe 2016, 20, 573-583). Briefly, 4.5×10⁴ cells were seeded in the absence of antibiotics, washed, and exposed to F. novicida at a multiplicity of infection of 0.1. Plates were centrifuged at 1,000×g for 30 minutes at room temperature, followed by incubation at 37° C. for 30 minutes. Cells were treated with 50 μg/mL gentamicin for 30 minutes to remove extracellular bacteria and washed thoroughly. Infected cells were then treated with the indicated concentrations of the drugs and cells were lysed immediately and 16 h post infection by the addition of Triton X-100 to 0.1%. Lysates were diluted in TSBC and plated for colony-forming unit enumeration. Experiments were performed with at least four technical and three biological replicates.

G. Activity of the compounds in Galleria mellonella: G. mellonella larvae were procured from Vanderhorst Wholesale (St. Marys, Ohio) at least one day prior to infection and stored in the dark at room temperature. Larvae weighing less than 200 mg or more than 300 mg were omitted from study. To evaluate toxicity of the derivatives, they were diluted to 100 μM in phosphate buffer saline (PBS) and 10 μL was injected in the distal left proleg with UltiCare syringes (31G 3/10 cc 6 mm). The control groups included larvae that received doses of PBS only or were left uninjected to determine mortality related to needle-induced trauma. To determine if the compounds block F. novicida pathogenicity, F. novicida was washed with PBS, resuspended to 10⁵ CFU/10 μL, and 10 μL was inoculated in the distal right proleg two hours prior to injection with 10 μL of 80 μM derivative in the distal left proleg. After injection, all larvae were incubated at 33° C. in 24-well plates, and larval mortality was recorded twice daily for the duration of the study. Larval survival was graphed with Kaplan-Meier analysis and statistical comparisons were performed using Log-rank (Mantel-Cox) test.

H. Determining the activity of drugs on F. novicida growth: An overnight F. novicida culture was diluted 5000-fold in TSBC, derivatives were added to 10 μM, and the culture was incubated at 37° C. while shaking. At the indicated times, samples were collected from the cultures, diluted in TSBC, and plated to enumerate viable bacteria. Experiments were performed with at least six technical and three biological replicates.

I. Statistical analysis and reproducibility: All data are representative of a sufficient number of technical replicates to provide the power required to detect the indicated statistical differences. Interesting data garnered from the screening experiments were validated with subsequent assays to rule out false positives. Outside of the screening experiments, all data presented are representative of at least three biological replicates. Graphing and statistical comparisons of quantified data were performed by using Prism software (GraphPad, version 10.1.0).

Many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

What is claimed is:

1. A tolfenpyrad derivative of Formula I, or a pharmaceutically acceptable salt or stereoisomer thereof: 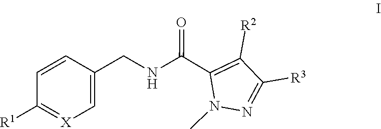
wherein:
X is CH or N;
R¹ is independently selected from the group consisting of C₁-C₁₀ alkyl, C₁-C₁₀ haloalkyl, C₃-C₁₀ cycloalkyl, C₁-C₁₀ alkoxy, 3-10 membered heterocycloalkyl, aryl, aryloxy, and heteroaryl;
R² and R³ are each independently selected from hydrogen, halogen, C₁-C₁₀ alkyl, C₂-C₁₀ alkenyl, C₂-C₁₀ alkynyl, C₁-C₁₀ haloalkyl, C₃-C₁₀ cycloalkyl, 4-10 membered heterocycloalkyl, aryl, aryloxy, and heteroaryl;
or,
R² and R³ are taken together with the carbons to which they are attached to form a C₃-C₁₀ cycloalkyl, 4-10 membered heterocycloalkyl, or heteroaryl; and,
R¹ cannot be 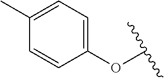 [SMILES: *Oc1ccc(C)cc1]
when R² is Cl and R³ is C₁-C₁₀ alkyl.

2. The derivative of claim 1, wherein R¹ is aryl and X is CH.

3. The derivative of claim 2, wherein R¹ is selected from the group consisting of:

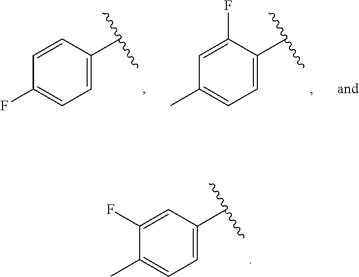

4. The derivative of claim 1, wherein R¹ is aryloxy and X is CH.

5. The derivative of claim 4, wherein R¹ is:

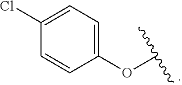 [SMILES: *Oc1ccc(Cl)cc1]

6. The derivative of claim 1, wherein R¹ is heteroaryl.

7. The derivative of claim 6, wherein R¹ is thiazole, thiadiazole, or oxadiazole.

8. The derivative of claim 7, wherein R¹ is selected from the group consisting of:

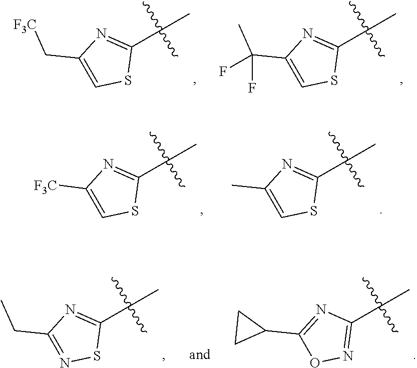

9. The derivative of claim 1, wherein R² is halogen or hydrogen.

10. The derivative of claim 9, wherein R² is Cl.

11. The derivative of claim 1 wherein R³ is C₁-C₁₀ alkyl or hydrogen.

12. The derivative of claim 11, wherein R³ is ethyl.

13. The derivative of claim 1, wherein R² and R³ together form a C₃-C₁₀ cycloalkyl.

14. The derivative of claim 13, wherein the cycloalkyl is cyclopentane.

15. The derivative of claim 1, wherein the compound is selected from the group consisting of:

| | |
|---|---|
| NK01 | 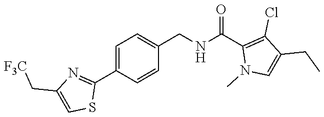 [SMILES: CCc1cn(C)c(C(=O)NCc2ccc(-c3nc(CC(F)(F)F)cs3)cc2)c1Cl] |
| NK02 | 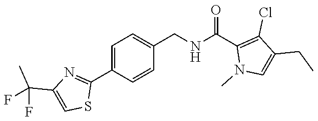 [SMILES: CCc1cn(C)c(C(=O)NCc2ccc(-c3nc(C(C)(F)F)cs3)cc2)c1Cl] |
| NK03 | 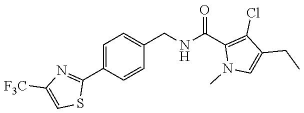 [SMILES: CCc1cn(C)c(C(=O)NCc2ccc(-c3nc(C(F)(F)F)cs3)cc2)c1Cl] |
| NK04 | 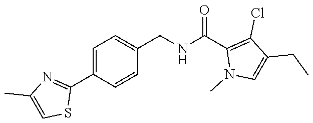 [SMILES: CCc1cn(C)c(C(=O)NCc2ccc(-c3nc(C)cs3)cc2)c1Cl] |
| NK05 | 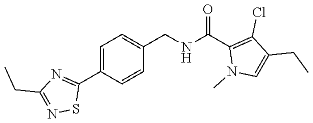 [SMILES: CCc1nsc(-c2ccc(CNC(=O)c3c(Cl)c(CC)cn3C)cc2)n1] |
| NK06 | 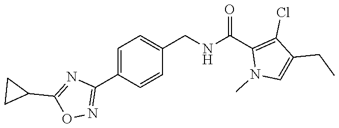 [SMILES: CCc1cn(C)c(C(=O)NCc2ccc(-c3noc(C4CC4)n3)cc2)c1Cl] |
| NK07 | 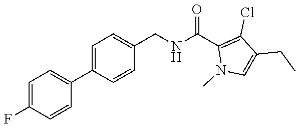 [SMILES: CCc1cn(C)c(C(=O)NCc2ccc(-c3ccc(F)cc3)cc2)c1Cl] |
| NK08 | 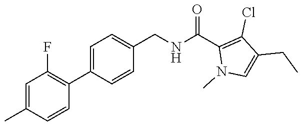 [SMILES: CCc1cn(C)c(C(=O)NCc2ccc(-c3ccc(C)cc3F)cc2)c1Cl] |
| NK09 | 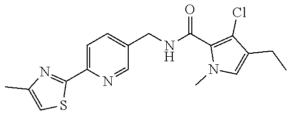 [SMILES: CCc1cn(C)c(C(=O)NCc2ccc(-c3nc(C)cs3)nc2)c1Cl] |
| NK10 | 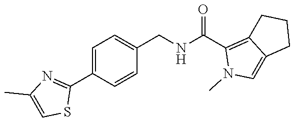 [SMILES: Cc1csc(-c2ccc(CNC(=O)c3c4c(cn3C)CCC4)cc2)n1] |
| NK11 | 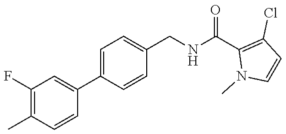 [SMILES: Cc1ccc(-c2ccc(CNC(=O)c3c(Cl)ccn3C)cc2)cc1F] |
| NK12 | 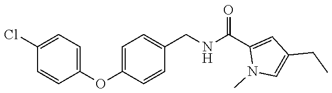 [SMILES: CCc1cc(C(=O)NCc2ccc(Oc3ccc(Cl)cc3)cc2)n(C)c1] |

or a pharmaceutically acceptable salt or stereoisomer thereof.

16. A pharmaceutical composition comprising at least one derivative of claim 1, or a pharmaceutically acceptable salt or stereoisomer thereof, and a pharmaceutically acceptable carrier.

17. A method of treatment of a disorder associated with a Francisella species bacterial pathogen, comprising administering a subject suffering from the disorder associated with the Francisella species bacterial pathogen an effective amount of at least one derivative of claim 1, or a pharmaceutically acceptable salt or stereoisomer thereof.

18. The method of claim 17, wherein the Francisella species is Francisella tularensis.

19. The method of claim 17, wherein the disorder is Tularemia.
